(19) United States
(12) Patent Application Publication
(10) Pub. No.: US 2021/0321649 A1
(43) Pub. Date: Oct. 21, 2021

(54) METHOD FOR DETECTING MISHANDLING AND MISUSE OF FOOD PRODUCTS

(71) Applicants: SECURITY MATTERS LTD., Hevel Eilot (IL); SOREQ NUCLEAR RESEARCH CENTER, Hevel Eilot (IL)

(72) Inventors: Nadav YORAN, Tel Aviv (IL); Tzemah KISLEV, Mazkeret Bathya (IL); Yair GROF, Rehovot (IL); Haggai ALON, Kibbutz Naan (IL); Mor KAPLINSKY, Herzliya (IL)

(73) Assignees: SECURITY MATTERS LTD., Hevel Eilot (IL); SOREQ NUCLEAR RESEARCH CENTER, Yavne (IL)

(21) Appl. No.: 16/336,712

(22) PCT Filed: Sep. 25, 2017
(86) PCT No.: PCT/IL2017/051073

§ 371 Date: Mar. 26, 2019

Related U.S. Application Data
(60) Provisional application No. 62/399,561, filed on Sep. 26, 2016.

(51) Int. Cl. A23L 5/30; A23L 13/00; A23L 15/00; A23L 29/00; A23L 29/30; B41M 3/06; A01K 43/10; G01N 33/08; G01N 33/12; G01N 23/223

(52) U.S. Cl. CPC A23L 5/36; A23L 13/00; A23L 15/00; A23L 29/015; A23L 29/045; A23L 29/30; A23V 2002/00; B41M 3/06; A01K 43/10; G01N 33/08; G01N 33/12; G01N 23/223; A23L 29/035

(57) ABSTRACT

Provided is a method for marking a product for human or animal use with an XRF identifiable mark, the method including forming on at least a region of the product a pattern of at least one FDA-grade material identifiable by XRF, the pattern being optionally at least partially invisible to the naked human eye and having a predefined identifiable characteristic, wherein the product is selected from food products, therapeutics and cosmetics.

### TECHNOLOGICAL FIELD

The invention generally concerns methods for marking products suited for human or animal consumption or use by markers comprising compounds which are identifiable by XRF.

### BACKGROUND

Product management protocols are typically appended to products suitable for human consumption or use in order to ensure product safety and minimize disruption of the conditions under which such products are to be maintained during manufacture, shipping, storage and generally handling prior to use. Where food products and therapeutic materials are concerned, such protocols dictate the maintenance of temperatures and exposure to sunlight, exposure to oxidizing materials and external stimuli which have an effect on the product integrity and eventual safety of use.

While the existing complex global supply chain allows consumers to buy fresh produce and food products, variations in temperature during shipment and non-standardized, non-continuous methods of control throughout the supply chain, increase the chance of end users consuming or using products that impose risk to their health.

X-ray fluorescence (XRF) has been utilized in the past for the purpose of identifying products based on XRF marking present on the product. In XRF the emission of characteristic “secondary” (or fluorescent) X-rays from a material that has been excited by primary X-rays or gamma rays radiation is detected. The term fluorescence refers to the absorption of radiation of a specific energy resulting in the re-emission of radiation of a different energy (typically lower). The XRF phenomenon is based on the fact that when materials are exposed to short-wavelength X-rays or gamma rays, they expel electrons from inner orbitals of the atom, which thus causes electrons in higher orbitals to “fall” into the lower/inner orbital, and, in the process, release photons with energy equal to the energy difference between the two orbitals involved.

Different chemical elements have electronic orbitals/shells of different characteristic energies, and therefore the spectral profile of an XRF response from an object/material is indicative of the chemical elements and possibly of the amount of each element included in the material/object.

Several publications are known to teach XRF marking and authentication of a variety of materials, including food products, medicinal products and cosmetic products [1-4]; however none of the known methods provides the necessary means for detecting tampered with food products or such products that have undergone undesired treatment during storage, handling or transportation or have otherwise been modified to eliminate or remove one or more indicators of freshness and usability. Thus, the need remains for a safe and robust methodology that would not only have the capability to monitor changes in the chill-chain of a product, such as food products, but also attempts at repackaging or otherwise tempering with the integrity of such products.

### REFERENCES

### [1] US 2003/0194052

[2] U.S. Pat. No. 8,864,038

### [3] US 2002/0173042

[4] U.S. Pat. No. 8,590,800.

### GENERAL DESCRIPTION

The invention disclosed herein concerns methods for marking products for human and animal use and packaging of same by XRF-detectable markers, which provide an indication to the product history, e.g., whether the product has been tampered with or undergone undesired treatment during storage, handling or transportation or has otherwise been modified to eliminate or remove one or more indications concerning its freshness and usability.

The technology disclosed herein is not only limited to the marking of products for consumption by humans or animals, but also to any other product which chain of handling, storage and transportation is to be closely monitored. Such products may additionally be pharmaceuticals, cosmetics, veterinary products and others.

The marking method is based on the application or addition of FDA-graded materials (or substances which may be consumed orally and in particular GRAS-grade or allowed for use as food coloring or flavor enhancers) to food products or food packaging, cosmetic products, and pharmaceutical products, thereby authenticating them, namely identifying the product with one or more of manufacturer details or identification code, date of manufacture, site of manufacture, date of expiration, etc. The marking may additionally or alternatively be tailored such that phase changes undergone previously by the product can be detected by detecting and measuring changes in constitution (composition and phase change), shape and position of the marker(s). In particular, the present method allows for detecting whether a food product, for example meat products, (whether in a frozen state or not) had previously undergone defrosting, or whether a marking, either an abstract marking or a special marking e.g., expiration or “use by” date, have been tampered with or reapplied to extend the date to a date later than a product expiration date.

Thus, in one aspect, there is provided a method for marking a product for human or animal use with an XRF pattern, the method comprising forming on at least a region of the product a pattern of at least one FDA-grade material identifiable by XRF, the pattern being optionally at least partially invisible to the naked human eye and having a predefined identifiable characteristic.

The invention further provides a method of authenticating a product for human or animal use, the method comprising forming at least one XRF identifiable mark on the product prior to commercialization, reading and storing for reference at least one XRF identifiable characteristic of said mark, and reading said mark at any point in time following commercialization of the product to identify the mark and determine whether the at least one XRF identifiable characteristic has undergo a change. A change in the characteristic of the mark at a point later to the date of commercialization, from that measured once the mark had been formed, indicating unauthorized treatment or misuse, e.g., tempering therewith.

The product to be marked by an XRF pattern is a product typically used by humans or animals and may be selected from food products, or generally products for human or animal consumption, therapeutics, cosmetics or any other product which misuse, mishandling or tampering may render its use by humans or animals unsafe.

In some embodiments, the product is a food product.

The mark may be formed on the product, e.g., food product, at any time during product manufacture or at any time prior to product commercialization. As used herein, “product commercialization” refers to any stage prior to final sale of the product, including storage, handling and transportation of said product. The product may be authenticated or analyzed for potential tampering at any time after the mark has been formed on the product or at any time after commercialization, namely after it has been stored, transported or generally handled prior to final sale.

The mark formed on a product, e.g., food product, is said to have a predefined characteristic that is identifiable by XRF and which permits a determination of, e.g., unauthorized treatment or misuse. The predefined characteristic may be selected from a chemical composition of the mark, concentration of one or more component contained in the composition, the mark structure, size, and shape. Where the mark is identifiable by its components, independent of any structural feature or shape thereof, the mark composition may be selected to contain one or more components that define the product's date of manufacture, the product manufacture site, the product expiry date and so forth. Thus, for example, where a product has been appended with a visible mark identifying, e.g., a certain expiry date, and an XRF mark identifiable to a certain different expiry date, the product may be said to have been tempered with.

Similarly, a product may be said to have been tempered with in case an original mark, independent of structure and composition, made of at least FDA-grade material that is identifiable by XRF, and having an original identifiable signal of a first intensity, changes such that the concentration of the FDA-grade material that is identifiable by XRF is reduced and thus affects a change in the original identifiable signal.

Where the XRF mark is a special mark, whether visible to the naked eye or not, it may be in the form of a logo or any mark identifying the product, its manufacturer, date of manufacture, date of expiry, “use by” date or any other textual or non-textual mark.

In some embodiments, the mark or pattern formed in/on the product is responsive to at least one external stimulus such as temperature, oxygen, moisture, mechanical treatment, repackaging, and others. In some embodiments, the pattern of the at least one FDA-grade material that is identifiable by XRF may be selected to have a predefined shape, size and material constitution (namely, which marker material is used and at what concentration), which change upon exposure to the external stimulus. In other words, the predefined pattern has a first characteristic that is selected as an authentication mark which would transition from its formed shape, size and constitution to a different unpredictable pattern upon any mishandling of the product.

Thus, the invention further provides a method for marking a product for human or animal use with an XRF pattern, the method comprising forming on at least a region of the product a pattern of at least one FDA-grade material identifiable by XRF, the pattern having a first characteristic being responsive to an external stimulus by transitioning from the first characteristic to a second characteristic, said transition being identifiable by XRF and indicative of exposure to said external stimulus.

The FDA-grade materials used in markers in accordance with the present invention are substances which include at least one compound or element identifiable by X-Ray Fluorescence (XRF) signature, namely can be identified by XRF analysis (e.g. by an energy dispersive X-ray Fluorescence analysis). The XRF signature can be identified by a suitable spectrometer such as an XRF analyzer. The XRF analyzer may be a mobile handheld device which may work in uncontrolled environment (e.g. without vacuum conditions), such as the XRF analyzer described in U.S. provisional Patent Application No. 62/396,412 which is incorporated herein by reference.

The markers utilized in accordance with the present invention may be soluble in polar or nonpolar solvents or may be insoluble once dried on the surface of a product or a packaging material.

The FDA-grade material may be any material or additive which may be added to the product without affecting the product constitution, stability, shelf-life, usability, toxicity, etc. The materials may be in the form of salts or organic materials, may be in the form of metal ions or metal-ligand forms or any combination thereof. In some embodiments, the FDA-grade material is a material having an XRF signature and selected amongst the so-called Generally Recognized As Safe (GRAS) materials, under sections 201(s) and 409 of the Federal Food, Drug, and Cosmetic Act (the Act), USA. The GRAS list is herein incorporated by reference.

The FDA-grade material may be in the form of a compound which includes one or more elements that are safe for human or animal use and at the same time identifiable by XRF. In some embodiments, the FDA-grade material is or comprises at least one element of the periodic table of the elements which in response to x-ray or gamma-ray (primary radiation) radiation emits an x-ray signal (secondary radiation) with spectral features (i.e. peaks in a particular energy/wavelength) characteristic of the element (an x-ray response signal as XRF signature). An element having such response signal is considered XRF-sensitive.

In some embodiments, the FDA-grade material is an element or a material comprising one or more elements, the element having an electronic transition between atomic energy levels which generates an identifiable x-ray signal.

In some embodiments, the material comprises at least one metal ion. In some embodiments, the FDA-grade material is an element selected from Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La and Ce. In other embodiments, the FDA-grade material is a material comprising one or more elements selected from Si, P, S, Cl, K, Ca, Br, Ti, Fe, V, Cr, Mn, Co, Ni, Ga, As, Fe, Cu, Zn, Ga, Rb, Sr, Y, Zr, Nb, Mo, Tc, Ru, Rh, Ag, Cd, In, Sn, Sb, Te, I, Cs, Ba, La and Ce.

In some embodiments, the FDA-grade material is at least one element selected from Cu, K, Zn, Ca, Co, Fe, Mg, Na and Li.

In some embodiments, the FDA-grade material is a marker combination comprising two or more elements; each may be presented in a different oxidation state; as different metal complex; different ligands; at different concentrations; or presented in different carriers within the marking composition.

In some embodiments, the FDA-grade material combination comprises a plurality of marker elements, each being present in different concentrations or form, to permit a unique signature with spectral features characteristic not only of the specific elements in the combination but also of their relative concentrations.

The FDA-grade material in a marker combination or independent of other markers in a composition may be in a metal form, a salt form, an oxide form, a polymer comprising (in a chemical or a physical interaction) said one or more elements, an organometallic compound, or a complex which includes one or more of the elements.

In some embodiments, the material is selected amongst organic materials.

Depending, inter alia, on the product to be marked and the site of marking, the FDA-grade material may be selected based on its solubility. For example, where the product is marked on its most external surface, the material may be selected to be soluble in an organic solvent. Where marking is to be affected on the product itself, e.g., on the food product, the material may be selected to be soluble in water.

In some embodiments, the FDA-grade material is a material that is not native to the product itself, namely it is not typically form part of the food product or its packaging material. In some cases, where the material is present in the product or its packaging, the material added as a marking material may be different from that present or in an amount that is not typically present in the product or its packaging.

In some embodiments, the FDA-grade material is selected from the materials listed in Table 1 below:

| Marking | | Solubility in | Solubility in |
|---|---|---|---|
| Element | Compound | Ethanol | water |
| Cu | Copper complexes | Soluble | Insoluble |
| | chlorophylls (E141) | | |
| Co | Vitamin B12 (4.3% | Soluble | Soluble |
| | Co) | | |
| Fe | Ferrous lactate | | Soluble |
| | (E585) | | |
| Fe | Ferrous gluconate | | Soluble |
| | (E579) | | |
| Fe | Ferrous ascorbate | | Soluble |
| Fe | Ammonium ferric | | Soluble |
| | citrate | | |
| Mn | Manganese citrate | | Soluble |
| Mn | Manganese | Slightly | Soluble |
| | gluconate | | |

The FDA-grade materials, being marker materials, may be formulated into ‘ink’ formulations or marking formulations or compositions comprising one or more of the marking materials, a liquid carrier and one or more additives. These ‘ink’ formulations are not limited in any way to one form of application or any way of forming the XRF-pattern. The markers may be added or mixed with the products in liquid or paste from or in minced or grounded state (e.g. minced meat product). Additionally, the markers can be mixed with the bulk material of the product or be applied onto to the surface of the product by printing (e.g. solid products such as eggs), stumping or pressing (e.g. on meat, and poultry products, and on fruits and vegetables), or sprayed onto the product. The markers may also be applied to packaging and labels. In addition, the markers may be added to various dyes and inks used for marking the products.

The concentrations or amounts of the FDA-grade material(s) in any marking composition of the invention may be set according to a preselected code, which can be measured by XRF analysis, after application of the composition onto the, e.g., food product, in the authentication stage. In general, the marking composition may include one or more FDA-grade materials with preselected concentrations within the range of 0.1 to 10,000 ppm; the preselected concentration may be adapted or preset to code for a specific product identification; a specific date relating to the product, such as date of manufacture, date of expiry, etc; site of manufacture; product content; and others. In some embodiments, the composition comprises at least one FDA-grade material in a concentration between 0.1 and 1,000 ppm, 0.1 and 900 ppm, 0.1 and 800 ppm, 0.1 and 700 ppm, 0.1 and 600 ppm, 0.1 and 500 ppm, 0.1 and 400 ppm, 0.1 and 300 ppm, 0.1 and 200 ppm, 0.1 and 100 ppm, 0.1 and 10 ppm, 0.1 and 9 ppm, 0.1 and 8 ppm, 0.1 and 7 ppm, 0.1 and 6 ppm, 0.1 and 5 ppm, 0.1 and 4 ppm, 0.1 and 3 ppm, 0.1 and 2 ppm, 0.1 and 1 ppm, 1 and 1,000 ppm, 1 and 900 ppm, 1 and 800 ppm, 1 and 700 ppm, 1 and 600 ppm, 1 and 500 ppm, 1 and 400 ppm, 1 and 300 ppm, 1 and 200 ppm, 1 and 100 ppm, 1 and 90 ppm, 1 and 80 ppm, 1 and 70 ppm, 1 and 60 ppm, 1 and 50 ppm, 1 and 40 ppm, 1 and 30 ppm, 1 and 20 ppm, 1 and 10 ppm, 1 and 9 ppm, 1 and 8 ppm, 1 and 7 ppm, 1 and 6 ppm, 1 and 5 ppm, 1 and 4 ppm, 1 and 3 ppm or 1 and 2 ppm.

In some embodiments, the composition comprises at least one FDA-grade material in a concentration sufficient for marking a single product. In such embodiments, the concentration may be suitable for obtaining a film on the surface of the product, on the surface of the packaging material surrounding the product, or on the immediate surface of the product, the film may be in a form as disclosed herein.

In some embodiments, the film comprising between about 0.1 and 1,000 ppm, 0.1 and 900 ppm, 0.1 and 800 ppm, 0.1 and 700 ppm, 0.1 and 600 ppm, 0.1 and 500 ppm, 0.1 and 400 ppm, 0.1 and 300 ppm, 0.1 and 200 ppm, 0.1 and 100 ppm, 0.1 and 10 ppm, 0.1 and 9 ppm, 0.1 and 8 ppm, 0.1 and 7 ppm, 0.1 and 6 ppm, 0.1 and 5 ppm, 0.1 and 4 ppm, 0.1 and 3 ppm, 0.1 and 2 ppm, 0.1 and 1 ppm, 1 and 1,000 ppm, 1 and 900 ppm, 1 and 800 ppm, 1 and 700 ppm, 1 and 600 ppm, 1 and 500 ppm, 1 and 400 ppm, 1 and 300 ppm, 1 and 200 ppm, 1 and 100 ppm, 1 and 90 ppm, 1 and 80 ppm, 1 and 70 ppm, 1 and 60 ppm, 1 and 50 ppm, 1 and 40 ppm, 1 and 30 ppm, 1 and 20 ppm, 1 and 10 ppm, 1 and 9 ppm, 1 and 8 ppm, 1 and 7 ppm, 1 and 6 ppm, 1 and 5 ppm, 1 and 4 ppm, 1 and 3 ppm or 1 and 2 ppm.

The concentration of the FDA-grade material may be determined based on the size of the product, the characteristics of the film or mark to be formed on the product and other parameters having to do with the stability of the marking composition. For any typical object, on average, the amount of FDA-grade material(s) may be between 0.1 and 10,000 ppm per product. The amount of the marker may be homogenously distributed on the surface of the product or may be distributed in material increments throughout the surface of the process. For example, where the amount of a FDA-grade material in a composition suitable for application onto a single product is 100 ppm, the composition may be applied such that 10 equal portions are applied onto 10 different regions of the product, each portion comprising 10 ppm of the marker material.

Markers, which are soluble in water, may be applied to a limited/localized area on the surface of the product or packaging at a preselected time before it is frozen or during the freezing process. For example, meat or poultry products can be stamped with ink which includes one or more markers prior to freezing. Similarly, the markers may be printed on fruits or vegetable or paper labels attached to food products which are to be frozen. As the food product is frozen (inside or outside a packaging) the markers will remain in the surface area on which they were initially applied indefinitely and maintain their shape and position on the product. However, once the food product is defrosted water droplets condense on the food product, wetting the surface and the markers. Since the markers are solvable in water, the defrosting process will spread the initially localized markers over a larger area, thereby decreasing its concentration on the initial marked area and increasing the concentration of the markers within the vicinity of the marked area (on previously unmarked surface areas). This affect can be measured using a reader (i.e. XRF analyzer) which can measure the concentration of the markers.

In certain products, such as meat or poultry products, the wetting of the marked surface area is caused also by the food product itself, wherein the frosting and defrosting process causes water-based or oil-based liquids initially retained within the product to be transferred to the surface as well. For example, in meat or poultry the frosting may damage the cellular walls, such that upon defrosting, water based liquids which in fresh meat are kept within the tissue are delivered to the surface of the meat, dissolving the markers.

Due to the irreversible process of dissolving and the spreading of the marker, the method of the present invention enables, by calibrating the XRF reader taking into account to the type of the food product and its surface, the measurement of the amount of time in which the food product has stayed in temperatures which are higher than a preselected threshold temperature.

The marking of the present invention can be applied by various methods in a precise and localized manner Namely, an area with relatively fine border lines as compared to the surface area which is read/measured by the XRF reader. Therefore, in detecting whether the food product had undergone a phase change (e.g. defrosted) one can measure whether the concentration of the markers in the marked area decreased, or measure whether the concentration of the markers in the vicinity of the marked area increased (indicating that the markers are spread over a larger area). One can also measure both areas for improving the accuracy of the measurement

The markers can be mixed with dyes and inks to provide a visible indication that the markers applied to a localized surface area of the food product had been dissolved and spread over a larger area.

The markers utilized in accordance with the present invention may be added or applied to a food product such that their concentration on the surface or in the bulk of the food product are set according to a preselected code. Therefore, information can be encoded by using the markers. In particular information relating to the chain of supply or the manufacturing process, such as the date of manufacture and/or date and in which a product was frozen or defrosted.

As stated above, the markers can be mixed in the bulk material of food product, cosmetic products or pharmaceutical products in liquid or paste form, or to such products in granular form or to product that have been grounded, by mechanical mixing or by spraying. Marking the bulk material of a product allows one to detect whether the product has been diluted or mixed with (unmarked) products of lesser quality or with undesirable additive. For example, liquid food products and in particular alcoholic products, cosmetic products such as perfumes or creams and pharmaceutical products.

In another example, granular or grounded food products such as minced meat can be sprayed with markers in an aerosol form, or alternatively be mechanically mixed with markers, during or after the mincing process. The marking of minced meat for example may include the information whether the minced meat is selected to be frozen or to be sold fresh. Such a marking can be used as a measure against the illegal practice (common in some countries) of mincing defrosted out-of-date meat together with fresh minced meat, since the concentration of the markers present in the fresh meat will decrease.

As stated above, the method of forming the pattern permits facile detection of any change in the pattern, namely any deviation from the predefined characteristic, e.g., the above recited first characteristic. Thus, where the first and second characteristics are different, the product may be said to have been mishandled or tampered with and thus may be unsafe for human or animal use. Where the first and second characteristics are the same, the product may be said to have been handled as intended. The sensitivity of the detection and the resolution of the measurement of the concentration of the marker can be increased by various methods for processing and enhancing the XRF signals received by the XRF reader, improving for instance the signal-to-noise ratio. For example, the methods described in International application PCT/IL2016/050340, incorporated herein by reference.

The invention thus provides a product having at least one XRF-identifiable marking associated therewith, the marking having a predefined characteristic, as a code, which identifies the product based on one or more of the following: chemical composition of the marking, concentration of any one component present in the marking, the marking position, the marking shape and others. The predefined characteristic may also depend on features of the marked product such as the shape, texture and/smoothness of its surface and parameters of the measurement of the XRF signal such as the distances between the surface of the product and the detector detecting the X-ray signal arriving from the product and/or the emitter irradiating the product, as well as the orientation product's surface relatively to the emitter and detector and others.

In some embodiments, the predefined characteristic is responsive to an external stimulus by transitioning from the predefined (first) characteristic to a second characteristic, said transition being identifiable by XRF and indicative of exposure to said external stimulus.

The invention further provides a method for determining mishandling of a product, the method comprising:
forming an XRF-identifiable pattern on the product, said pattern having a first characteristic responsive to an external stimulus;
irradiating said product with said X-Ray or Gamma-Ray radiation;
detecting an X-Ray signal arriving from the product in response to the X-Ray or Gamma-Ray radiation applied to the product;
applying spectral processing to the detected X-Ray signal to obtain data indicative of the presence, absence or any change in the first characteristic.

The XRF method may further comprise: (i) filtering a wavelength spectral profile of a detected portion of the X-Ray signal arriving from the product in response to X-Ray or Gamma-Ray radiation applied to the product to suppress trend and periodic components from the wavelength spectral profile to thereby obtain a filtered profile; and (ii) identifying one or more peaks in the filtered profiled satisfying a predetermined characteristic thereby enabling utilizing wavelengths of the one or more peaks to identify signatures of materials included in the product.

In some embodiments, the method comprises irradiating the product with the X-Ray or Gamma-Ray radiation; detecting a portion of an X-Ray signal arriving from the product in response to the radiation applied to the product; and applying spectral processing to the detected X-Ray signal to obtain data indicative of wavelength spectral profile thereof within a certain X-Ray band.

In some embodiments, the wavelengths and optionally also the magnitudes of the one or more peaks are used to determine material data indicative of types and possibly also concentrations of materials included in the product and thereby any changes or misuse the product has undergone.

Thus, in one aspect the invention provides a method for marking a product for human or animal use with an XRF identifiable mark, the method comprising forming on at least a region of the product a pattern of at least one FDA-grade material identifiable by XRF, the pattern being optionally at least partially invisible to the naked human eye and having a predefined identifiable characteristic, wherein the product is selected from food products, therapeutics and cosmetics.

Further provided is a method of authenticating a product for human or animal use, the method comprising forming at least one XRF identifiable mark on the product prior to commercialization, reading and storing for reference at least one XRF identifiable characteristic of said mark, and reading said mark at any point in time following commercialization of the product to identify the mark and determine whether the at least one XRF identifiable characteristic has undergo a change, wherein the product is selected from food products, therapeutics and cosmetics.

The invention further provides a method for marking a product selected from food products, therapeutics and cosmetics with an XRF pattern, the method comprising forming on at least a region of the product a pattern of at least one FDA-grade material identifiable by XRF, the pattern having a first characteristic being responsive to an external stimulus by transitioning from the first characteristic to a second characteristic, said transition being identifiable by XRF and indicative of exposure to said external stimulus.

In some embodiments of all methods of the invention, the product is a food product.

In some embodiments of all methods of the invention, the FDA-grade material is at least one GRAS material.

In some embodiments, the material comprises at least one metal ion, optionally selected from Cu, K, Zn, Ca, Co, Fe, Mg, Na and Li.

The invention further provides an ink formulation comprising at least one FDA-grade material and at least one carrier, the at least one FDA-grade material being identifiable by XRF, optionally for application onto at least one food product.

Thus a method is provided for determining mishandling of a product, the method comprising:
forming an XRF-identifiable pattern on the product, said pattern having a first characteristic responsive to an external stimulus;
irradiating said product with said X-Ray or Gamma-Ray radiation;
detecting an X-Ray signal arriving from the product in response to the X-Ray or Gamma-Ray radiation applied to the product;
applying spectral processing to the detected X-Ray signal to obtain data indicative of the presence, absence or any change in the first characteristic.

The invention further provides a method for marking an egg for human use with an XRF identifiable mark, the method comprising forming on at least a region of the egg a pattern of at least one FDA-grade material identifiable by XRF, the pattern being optionally at least partially invisible to the naked human eye and having a predefined identifiable characteristic.

A method of authenticating an egg for human use is further provided, the method comprising forming at least one XRF identifiable mark on the egg prior to commercialization, reading and storing for reference at least one XRF identifiable characteristic of said mark, and reading said mark at any point in time following commercialization of the egg to identify the mark and determine whether the at least one XRF identifiable characteristic has undergo a change.

Also provided is a method for marking an egg with an XRF pattern, the method comprising forming on at least a region of the egg a pattern of at least one FDA-grade material identifiable by XRF, the pattern having a first characteristic being responsive to an external stimulus by transitioning from the first characteristic to a second characteristic, said transition being identifiable by XRF and indicative of exposure to said external stimulus.

In some embodiments of all methods of the invention, the mark is formed from a solution comprising between 1 and 30% of a metal selected from Cu, Co, Fe and Mn. The metal may be presented as a complex or a metal ion.

The invention further provides a method of authenticating a meat product for human use, the method comprising forming at least one XRF identifiable mark on the product prior to commercialization, reading and storing for reference at least one XRF identifiable characteristic of said mark, and reading said mark at any point in time following commercialization of the product to identify the mark and determine whether the at least one XRF identifiable characteristic has undergo a change.

In some embodiments, the mark is formed from an aqueous solution comprising 10%-20% carrier such as soy protein and a marker, being optionally zinc chloride.

In some embodiments, the marker is present at a concentration of 1% and is optionally placed on the meat product prior to freezing.

### DETAILED DESCRIPTION OF EMBODIMENTS

### Example 1: Marking Meat Products

For the purpose of marking meat, a solution comprising 10%-20% soy protein (in flour form) is dissolved in water as a binder for binding zinc-chloride, the marker, to the meat. The marker is added to the solution in a concentration of 1%.

The solution is applied to fresh meat before freezing by stamping using a rubber stamp. The solution binds to the surface of the meat tissue. The meat then undergoes freezing and the solution remains as is on the surface of the meat while frozen. The concentration of the zinc and/or the chloride components of the marker can be read by X-ray Fluorescence analysis. Once the meat is de-frosted the solution becomes diluted, the zinc-chloride marker travels to previously un-marked areas in vicinity to the marked area. Consequently, the concentration of the zinc and/or the chloride in the mark increases in those areas and reduces in the originally marked area. These changes in concentrations of one or both marker components can be read by XRF analysis.

Alternative markers which can be used in the same soy-protein solution is zinc-citrate.

### Example 2: Additional Solution that can be Used for Marking Meat

The markers used in the previous example, i.e., zinc-chloride and zinc-citrate may alternatively be added to High Fructose Corn Syrup (HFCS) (a sweetener made from corn starch). The solution contains about 1% of the marker dissolved in the HFCS.

### Example 3: A Solution for Detecting Temperature Changes in Deep-Frozen Food Products

A solution of Propylene glycol (E1520) in water, in concentrations in the range of 10% to 60% are used. Propylene glycol is used as an anti-freeze agent. The melting/freezing temperature of the solution is set according to the concentration of the Propylene glycol as shown in Table 2 below:

TABLE 2

| Concentration of | Melting |
|---|---|
| propylene-glycol | Temperature |
| [%] | [° C.] |
| 0 | 0 |
| 10 | −3 |
| 20 | −7 |
| 30 | −12 |
| 36 | −18 |
| 40 | −20 |
| 43 | −23 |
| 48 | −29 |
| 52 | −34 |
| 55 | −40 |
| 60 | −51 |

For example, a solution comprising 36% propylene-glycol and 1% zinc-chloride (or alternatively zinc-citrate) in water can be used to mark a meat product that is to be transported at a temperature of −18° C. The mark is applied to the meat while fresh by various means, e.g., rubber stamp, and is then frozen at −18° C. The concentration of the marker on the stamped area and in the vicinity of the stamped area is then read and stored for reference. In case the temperature of the meat rises above −18° C., e.g., under improper transportation conditions, the solution will undergo phase transition to a liquid. In liquid form the solution will travel at least to the vicinity of the originally marked area, reducing the concentration of the marker in the originally marked area and increasing concentration in the vicinity of that area. These changes in the concentrations of the marker are measurable by XRF analysis. By adjusting the concentration of the propylene-glycol in the solution one can adjust the melting temperature of the solution. Therefore, using this method one can detect whether the temperature during storing or transportation deviated from a preselected temperature.

To the above solutions food coloring such as (E102 and E103) can be added in concentration of about 10% to make the marking visible.

### Example 4: Marking Eggs

For the purpose of marking eggs one or more of the compounds of Table 1, in concentrations of 1%-30%, may be added to mixtures of water and alcohol or to commonly used water based or alcohol based egg-inks (usually comprising a mixture of water, alcohol and organic pigments). For example, a solution of up to 10% Copper complexes chlorophylls (E141) which is used as a green food coloring can be added water and alcohol mixture or to egg inks comprising water, alcohol and organic pigment. The solution can be printed on the egg shell in commonly used egg-printing devices, egg-stamping machines or by manual printing. The markers (Copper complexes chlorophylls) can be detected using XRF analysis and therefore may be used for example for authenticating the origin of the egg. By mixing the marker (E 141) in a plurality of concentration and/or by adding additional compounds of Table 1 to the water/alcohol mixture a code can be introduced into the solution which can indicate, for example, the date of production of the egg, as well as additional information.

1. A method for marking a product for human or animal use with an XRF identifiable mark, the method comprising forming on at least a region of the product a pattern of at least one FDA-grade material identifiable by XRF, the pattern being optionally at least partially invisible to the naked human eye and having a predefined identifiable characteristic, wherein the product is selected from food products, therapeutics and cosmetics.

2. A method of authenticating a product for human or animal use, the method comprising forming at least one XRF identifiable mark on the product prior to commercialization, reading and storing for reference at least one XRF identifiable characteristic of said mark, and reading said mark at any point in time following commercialization of the product to identify the mark and determine whether the at least one XRF identifiable characteristic has undergo a change, wherein the product is selected from food products, therapeutics and cosmetics.

3. A method for marking a product selected from food products, therapeutics and cosmetics with an XRF pattern, the method comprising forming on at least a region of the product a pattern of at least one FDA-grade material identifiable by XRF, the pattern having a first characteristic being responsive to an external stimulus by transitioning from the first characteristic to a second characteristic, said transition being identifiable by XRF and indicative of exposure to said external stimulus.

4. The method according to claim 1, wherein the product is a food product.

5. The method according to claim 1, wherein the FDA-grade material is at least one GRAS material.

6. The method according to claim 5, wherein the material comprises at least one metal ion.

7. The method according to claim 6, wherein the metal ion is selected from Cu, K, Zn, Ca, Co, Fe, Mg, Na and Li.

8. An ink formulation comprising at least one FDA-grade material and at least one carrier, the at least one FDA-grade material being identifiable by XRF.

9. The ink formulation according to claim 8, for application onto at least one food product.

10. A method for determining mishandling of a product, the method comprising:
forming an XRF-identifiable pattern on the product, said pattern having a first characteristic responsive to an external stimulus;
irradiating said product with said X-Ray or Gamma-Ray radiation;
detecting an X-Ray signal arriving from the product in response to the X-Ray or Gamma-Ray radiation applied to the product;
applying spectral processing to the detected X-Ray signal to obtain data indicative of the presence, absence or any change in the first characteristic.

11. A method for marking an egg for human use with an XRF identifiable mark, the method comprising forming on at least a region of the egg a pattern of at least one FDA-grade material identifiable by XRF, the pattern being optionally at least partially invisible to the naked human eye and having a predefined identifiable characteristic.

12. A method of authenticating an egg for human use, the method comprising forming at least one XRF identifiable mark on the egg prior to commercialization, reading and storing for reference at least one XRF identifiable characteristic of said mark, and reading said mark at any point in time following commercialization of the egg to identify the mark and determine whether the at least one XRF identifiable characteristic has undergo a change.

13. A method for marking an egg with an XRF pattern, the method comprising forming on at least a region of the egg a pattern of at least one FDA-grade material identifiable by XRF, the pattern having a first characteristic being responsive to an external stimulus by transitioning from the first characteristic to a second characteristic, said transition being identifiable by XRF and indicative of exposure to said external stimulus.

14. The method according to claim 11, wherein the mark is formed from a solution comprising between 1 and 30% of a metal selected from Cu, Co, Fe and Mn.

15. The method according to claim 14, wherein the metal is presented as a complex or a metal ion.

16. A method of authenticating a meat product for human use, the method comprising forming at least one XRF identifiable mark on the product prior to commercialization, reading and storing for reference at least one XRF identifiable characteristic of said mark, and reading said mark at any point in time following commercialization of the product to identify the mark and determine whether the at least one XRF identifiable characteristic has undergo a change.

17. The method according to claim 16, wherein the mark is formed from an aqueous solution comprising 10%-20% soy protein and a marker, being optionally zinc chloride.

18. The method according to claim 17, wherein the marker is present at a concentration of 1%.

19. The method according to claim 16, wherein the mark is placed on the meat product prior to freezing.
